(19) United States
(12) Plant Patent Application Publication
(10) Pub. No.: US 2018/0177094 P1
(43) Pub. Date: Jun. 21, 2018

(54) Chrysanthemum plant named 'Zanmubonita Yellow'

(50) ;

(71) Applicant: Van Zanten Breeding BV, Valkenburg (NL)

(72) Inventor: Henricus Cornelius Maria Jacobs, Valkenburg (NL)

(73) Assignee: Van Zanten Breeding BV, Valkenburg (NL)

(21) Appl. No.: 15/530,245

(22) Filed: Dec. 16, 2016

(51) Int. Cl. A01H 5/02

(52) U.S. Cl. PLT/295

(57) ABSTRACT

A new and distinct chrysanthemum plant named ‘Zanmubonita Yellow’ is disclosed, it is characterized by its medium sized, double type blooms with yellow color ray florets and response time of 7 weeks. The new plant is normally used for commercial cut flower production.

### BACKGROUND OF THE INVENTION

Botanical designation: Chrysanthemum x morifolium Ramat.

Cultivar denomination: ‘Zanmubonita Yellow’.

### RELATED CULTIVARS

‘Zanmubonita Yellow’ is a mutant plant of ‘Zanmubonita’(unpatented).

### BACKGROUND OF THE INVENTION

The present invention comprises a new and distinct cultivar of chrysanthemum, botanically known as Chrysanthemum x morifolium, and referred to by the denomination ‘Zanmubonita Yellow’.

‘Zanmubonita Yellow’ is a product of a breeding and selection program to produce new chrysanthemum cultivars for cut flower commercial applications. The new plant cultivar is a naturally whole plant mutant of a parent chrysanthemum named ‘Zanmubonita’ (unpatented). A comparison with the parent is made in the section ‘description of the invention’. The new cultivar was discovered as a whole plant mutation in June 2014 by Hencirus Jacobus Maria Jacobs in a controlled environment (greenhouse) in Valkenburg Z-H, The Netherlands. The first act of asexual reproduction of ‘Zanmubonita Yellow’ was accomplished when vegetative cuttings were taken from the initial selection and propagated further in Valkenburg Z-H, The Netherlands.

### SUMMARY OF THE INVENTION

The present invention is a new and distinct variety of a cut type chrysanthemum bearing medium sized double type blooms with yellow ray-florets and a response time of seven weeks.

### BRIEF DESCRIPTION OF THE DRAWINGS

The present invention of a new and distinct variety of chrysanthemum (of about 10 weeks) is shown in the accompanying drawings, the color being as nearly true as possible with color photographs of this type.

FIG. 1 shows a plant of the cultivar in full bloom.

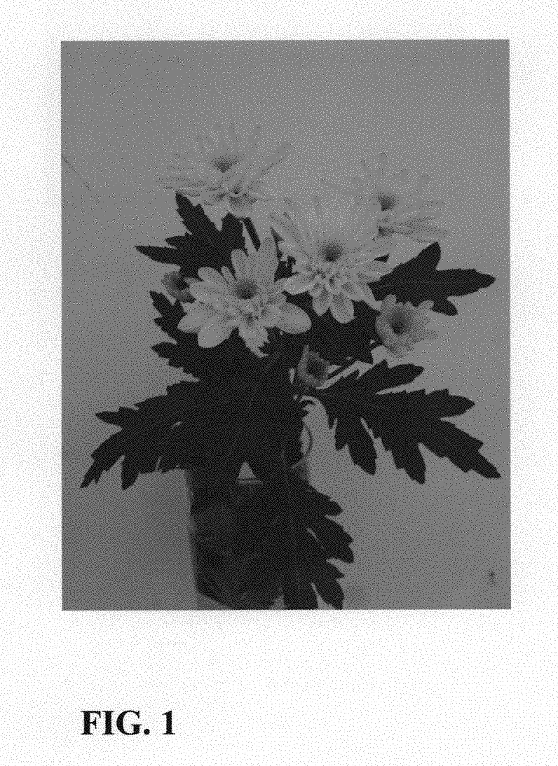

FIG. 2 shows the various stages of bloom of the new cultivar.

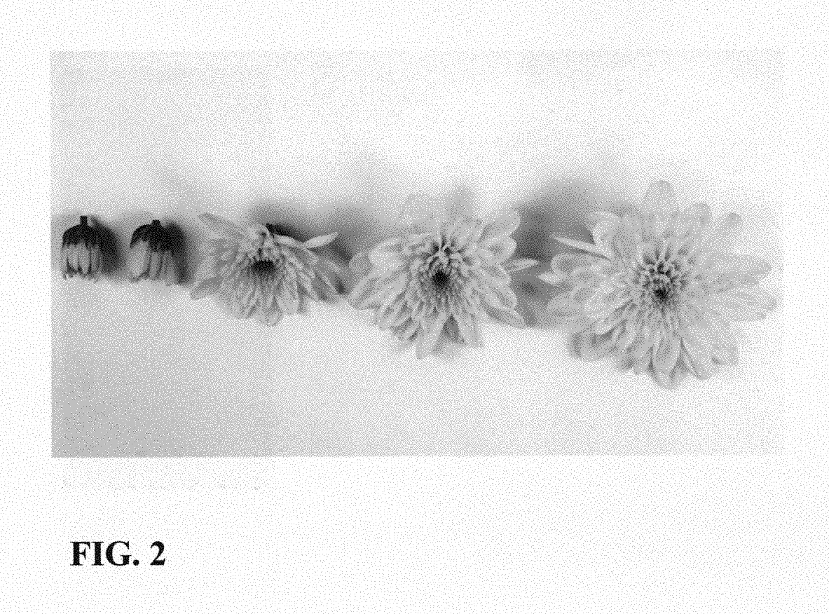

FIG. 3 shows the foliage of the new cultivar.

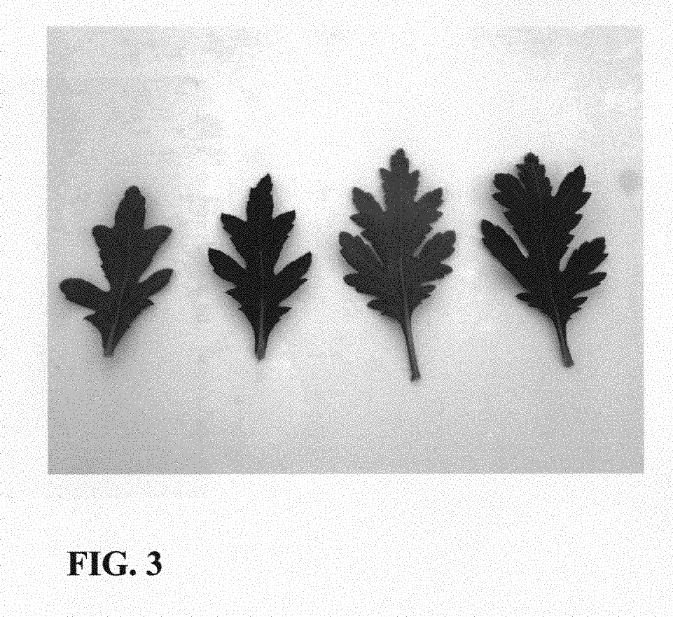

### DESCRIPTION OF THE INVENTION

The observations and measurements were gathered from plants grown in August to September 2016 in a greenhouse in Valkenburg Z-H, the Netherlands in a photo-periodic controlled crop under conditions generally used in commercial practice. The greenhouse temperatures during this crop were at day-time between 18° C. and 25° C. and at night 20° C. After a long day period of 14 days the photo-periodic response time in this crop was 49 days. After the long day period to flowering growth retardants (Alar) were applied 2 to 3 times in an average dose of 2.5 gram/liter water. No tests were done on disease or insect resistance or susceptibility. No tests were done on cold or drought resistance. This new variety of chrysanthemum has been found to retain its distinctive characteristics throughout successive propagations however the phenotype may vary significantly with variations in environment such as light intensity and temperature.

From the cultivars known to inventor the most similar existing cultivar in comparison to ‘Zanmubonita Yellow’ is ‘Zanmubonita’. When' Zanmubonita’ and Zanmubonita Yellow’ are being compared the following differences and similarities are noticed: The difference of ‘Zanmubonita’ and ‘Zanmubonita Yellow’ is ray-floret color. For which characteristic Zanmubonita Yellow has been selected out of Zanmubonita. All other characteristics of ‘Zanmubonita’ and ‘Zanmubonita Yellow’ are similar.

The following is a description of the plant and characteristics that distinguish Zanmubonita Yellow as a new and distinct variety.

The color designations are taken from the plant itself. Accordingly, any discrepancies between the color designations and the colors depicted in the photographs are due to photographic tolerances. The color chart used in this description is: The Royal Horticultural Society Colour Chart, edition 2001.

TABLE 1
Botanical Description of Chrysanthemum × morifolium
Ramat. ‘Zanmubonita Yellow’
Bud

| | |
|---|---|
| Size: | Medium; cross-section 8 mm, |
| | height 7 mm |
| Shape: | Round |
| Texture: | Pubescent |
| Outside color: | Greyed-green 191A |

Phyllaries

| | |
|---|---|
| Number: | 24, arranged in 3 rows |
| Shape: | Elliptic |
| Apex: | Acute |
| Base: | Truncate |
| Margin: | Entire |
| Color: | Upper side: 136A |
| | Under side: Green 137A |
| Length and width: | 10 mm; 3.5 mm |
| Texture: | Pubescent |

Inflorescences

| | |
|---|---|
| Spray Form: | Cylindrical |
| Inflorescence Type: | Double |
| Inflorescence Height: | 2 cm |
| Inflorescence Diameter: | 7-8 cm |
| Peduncle length: | 10-17 cm |
| Peduncle color: | Green 138A |
| Peduncle diameter: | 2 mm |
| Peduncle texture: | Pubescent |
| Number per branch: | Approx. 16-17 inflorescences |
| Seeds: | Not observed. |
| Fragrance: | Fainy chrysanthemum odor |

Color inflorecences
Center of inflorescence: Immature stage: Yellow-green 143C;
Mature stage: Yellow-green 144C
Color of upper side of ray florets: Yellow 5C
Color of lower side of ray florets: Yellow 5C
Tonality from distance: A cut mum with yellow blooms and a green center
Color of ray florets after aging of the plant: Yellow 5C
Ray florets

| | |
|---|---|
| Number of types: | One |
| Type: | Ligulate |
| Texture: | Upper and under side smooth |
| Number: | 100-110 |
| Shape: | Elliptic |
| Apex: | Rounded |
| Base: | Acute |
| Cross-section: | Weakly concave |
| Longitudinal axis: | Straight |
| Length of corolla tube: | 2 mm |
| Ray floret margin: | Entire |
| Ray floret length: | 1.8-4 cm |
| Ray floret width (outer row): | 4-12 mm |
| Ratio length/width: | Medium |

Disc florets

| | |
|---|---|
| Disc diameter: | 1 mm |
| Distribution of disc florets: | Very few, only visible in |
| | mature stage of inflorescence |
| Shape: | Tubular |
| Color: | Yellow-green 145C at base to |
| | Green Yellow 143C at apex |
| Length: | 4 mm |
| Diameter: | 0.5 mm |

Receptacle:

| | |
|---|---|
| Color: | Yellow-green 145D |
| Shape: | Domed raised |
| Height: | 5 mm |
| Diameter: | 6 mm |

Reproductive Organs

| | |
|---|---|
| Androecium: | Present on only disc florets |
| Stamen length: | 3 mm |
| Stamen color: | Yellow-green 144A |
| Anther color: | Yellow 2A |
| Pollen: | Not observed |
| Gynoecium: | Present on both ray and disc |
| | florets |
| Style color: | Yellow-green 154C |
| Style length: | 4 mm (bij GM) |
| Stigma color: | Yellow 7A |
| Stigma width: | 1 mm |
| Ovary: | Enclosed in calyx |

Plant

| | |
|---|---|
| Growth habit: | Upright |
| Growth rate: | Medium |
| Height: | 100-110 cm |
| Width: | 25 cm |
| Stem color: | Green 138B |
| Stem texture: | Smooth, slightly pubescent |
| Stem diameter: | 6-7 mm |
| Stem anthocyanin coloration: | absent |
| Internode length: | 1.8-2 cm |
| Response time: | 49 days |
| Roots: | Short, fibrous |

Foliage

| | |
|---|---|
| Glossiness: | Weakly |
| Leaf color: | Upper side: Green 136A; |
| | Under side: Green 137C |
| Color midvein: | Upper side: Yellow-green |
| | 147D; |
| | Under side: Yellow-green |
| | 148D |
| Size: | Medium; length 10-12 cm, |
| | width 4.5-6.5 cm |
| Quantity per stem: | 26-30 |
| Shape: | Elliptic |
| Texture upper side: | Sparsely pubescent |
| Texture under side: | Pubescent |
| Venation arrangement: | Pinnate |
| Shape of margin: | Serrated |
| Shape of base of sinus between lateral lobes: | Rounded |
| Margin of sinus between lateral lobes: | Diverging |
| Shape of base: | Attenuate to Truncate |
| Apex: | Pointed |
| Petiole length: | 2 cm |
| Petiole diameter: | 2.5-3 mm |
| Petiole attitude: | Moderately upwards |
| Petiole color: | Yellow-green 147D |
| Stipules: | Small |

TABLE 2

| Differences with the comparison variety, when grown under | | |
|---|---|---|
| identical conditions | | |
| | ‘Zanmubonita Yellow’ | ‘Zamubonita’ |
| Ray-floret color | Yellow 5C | White NN155D |

I claim:

1. A new and distinct chrysanthemum plant named ‘Zanmubonita Yellow’ as described and illu- strated.
